(19) United States
(12) Patent Application Publication
(10) Pub. No.: US 2020/0087575 A1
(43) Pub. Date: Mar. 19, 2020

(54) METHOD AND SYSTEM FOR PROCESSING OIL SANDS AND OTHER MATERIALS WITH LOW ENVIRONMENTAL IMPACTS

(71) Applicant: Lakes Environmental Research Inc., Waterloo (CA)

(72) Inventors: Jesse Thé, Waterloo (CA); Roydon Andrew Fraser, Kitchener (CA)

(73) Assignee: Lakes Environmental Research Inc., Waterloo (CA)

(21) Appl. No.: 16/570,303

(22) Filed: Sep. 13, 2019

(30) Foreign Application Priority Data
Sep. 14, 2018 (CA) 3017560

(51) Int. Cl. C10G 1/02; C10G 1/00; C07C 29/151; C10G 2/00; C10J 3/72

(52) U.S. Cl. CPC C10G 1/02; C10J 2300/1861; C07C 29/1518; C10G 2/31; C10J 3/72; C10G 2300/4081; C10J 2300/1246; C10J 2300/1659; C10G 2300/1033; C10G 2300/1003; C10G 2400/26; C10G 2400/02; C10G 2400/04; C10G 2400/08; C10G 2400/06; C10J 2300/0943; C10J 2300/1665; C10J 2300/1807; C10G 1/002

(57) ABSTRACT

A method of processing a first material including an oil source, and a second material including a medium. The method includes mixing the first material and the second material to provide a blended feedstock mixture including predetermined respective proportions of the first material and the second material, and also including water. The blended feedstock mixture is heated in a pre-distillation process and is further heated in a distiller to at least partially crack and vaporize the oil source, to provide atmospheric gas oil and vacuum gas oil from the oil source, coked medium material including carbon-heavy hydrocarbons and sand, and a first barren hot medium material. The coked medium material is heated in a gasifier to provide a second barren hot medium material and syngas. Heat energy from certain products resulting from such heating is transferred to the blended feedstock mixture.

### FIELD OF THE INVENTION

The present invention is a method and a system for processing oil sands and other materials with low environmental impacts.

### BACKGROUND OF THE INVENTION

As is well known in the art, the typical systems and methods for processing oil sands that have been mined are relatively complex, and require significant water and energy inputs. In particular, the known processes involve the use and contamination of large volumes of water and the creation of large waste (tailings) ponds. Large volumes of CO₂ emissions (and emissions of other gases, e.g., NOₓ, SOₓ, and H₂S) are generated by heating the large volumes of water by combustion of fossil fuels, to the extent that oil sands processing has become a major contributor of CO₂ emissions. Because the conventional systems and methods typically involve transporting oil sands over relatively large distances, additional CO₂ emissions are realized.

“Dilbit”, which includes bitumen and a diluent such as naphtha, is conventionally produced from the oil sands at or near the mine and transported over relatively large distances to a refinery, via pipeline. In addition to the costs incurred in transportation of relatively unrefined materials and the diluent, the current practices impose a substantial risk of environmental damage, i.e., in the event of a leak from the pipeline.

The oil sands tailings ponds result in environmental pollution and degradation to a significant extent. Various methods of disposing of the tailings and eliminating the tailings ponds (to permit reclamation) have been proposed. However, the known methods would involve intensive use of energy and financial resources.

Yet another issue is the difficulties encountered in processing oil sands having relatively low bitumen content. Typically, the processing facilities are designed for oil sands having a preselected minimum bitumen content. However, when oil sands material is mined that has a bitumen content that is lower than the preselected minimum, the processing thereof in the conventional processing facilities is unsatisfactory, and uneconomic.

Certain other sources of petroleum products also require processing that, using know methods, results in environmental degradation. The typical systems for processing oil shale involve generation of significant amounts of CO₂. Oil shales are sedimentary rock that include kerogen, which may be processed to form petroleum products.

In addition, significant amounts of waste materials having high carbon content have accumulated. For instance, large amounts of petcoke (petroleum coke), which is a byproduct from processing bitumen to form petroleum, are produced, to create another polluting material. There are also other high-carbon content waste materials (e.g., certain plastic products), that have accumulated, to the extent that they have become serious environmental problems.

### SUMMARY OF THE INVENTION

There is a need for a system and a method of processing oil sands materials and other materials with low environmental impacts that overcomes or mitigates one or more of the disadvantages or defects of the prior art. Such disadvantages or defects are not necessarily included in those listed above.

In its broad aspect, the invention provides a method of processing a first material including an oil source, and a second material including a medium. The method included mixing the first and second materials to provide a blended feedstock mixture that includes water.

In a pre-distiallation process, the blended feedstock mixture is heated to between approximately 100° C. and approximately 150° C., to produce steam from the water and to vaporize light hydrocarbons from the oil source. Subsequently, in a distillation process, the blended feedstock mixture is further heated to between approximately 535° C. and approximately 600° C., to provide (i) atmospheric gas oil and (ii) vacuum gas oil from the oil source, (iii) a coked medium material that includes carbon-heavy hydrocarbons and the medium, and (iv) a first barrent hot medium material. The coked medium material is heated to between approximately 700° C. and approximately 800° C.

In a gasification process, the coked medium material is heated to between approximately 850° C. and approximately 1,000° C., to produce a second barren hot medium material and syngas, which includes hydrogen and carbon monoxide.

Heat energy is transferred from the first and second barren hot medium materials to certain materials, at certain points in the method of the invention. For instance, heat energy is transferred from at least a portion of the first barren hot medium material to the blended feedstock mixture in the pre-distillation process. In addition, or alternatively, heat energy is transferred from the second barrent hot medium material to the blended feedstock mixture in the distillation process. One or more of air and oxygen is injected into the gasification process, to promote at least partial oxidation of the coked medium material therein.

### BRIEF DESCRIPTION OF THE DRAWINGS

The invention will be better understood with reference to the attached drawings in which:

FIG. 1 is a block diagram illustrating an embodiment of a method of the invention;

FIG. 2 is a block diagram illustrating another embodiment of the method of the invention;

FIG. 3 is a block diagram illustrating another embodiment of the method of the invention;

FIG. 4 is a block diagram illustrating another embodiment of the method of the invention;

FIG. 5 is a block diagram illustrating another embodiment of the method of the invention;

FIG. 6 is a block diagram illustrating another embodiment of the method of the invention;

FIG. 7 is a block diagram illustrating another embodiment of the method of the invention; and

FIG. 8 is a block diagram illustrating another embodiment of the method of the invention.

### DETAILED DESCRIPTION

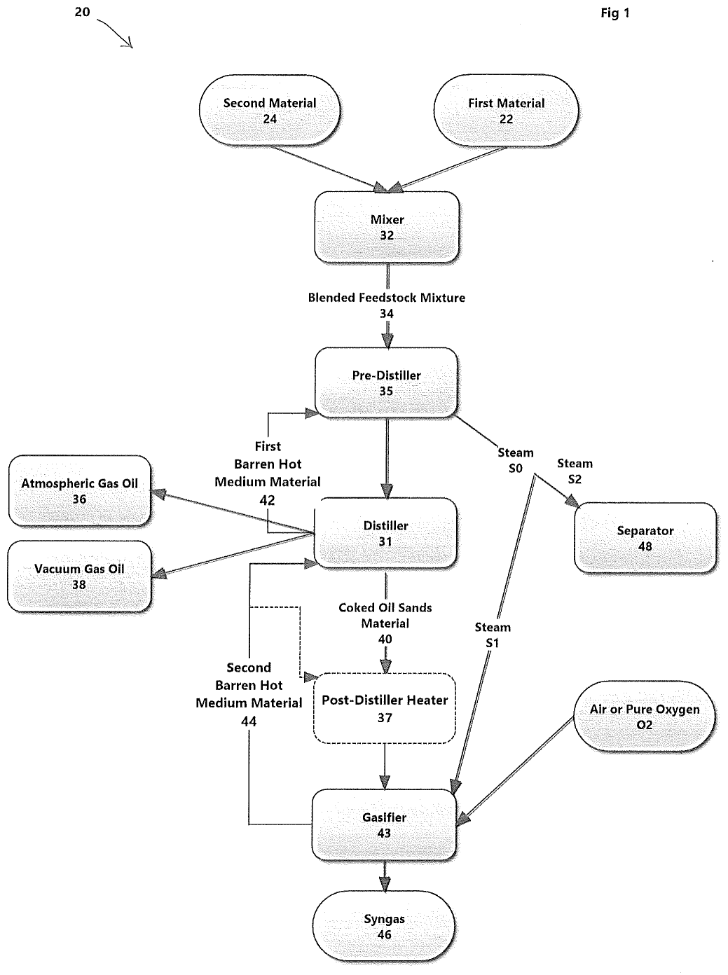

In the attached drawings, like reference numerals designate corresponding elements throughout. Reference is made to FIG. 1 to describe an embodiment of the method of the invention schematically represented therein that is indicated generally by the numeral 20. As will be described, in one embodiment, the method of the invention preferably is implemented via a system that includes a number of elements, schematically represented in the drawings.

As will also be described, the method 20 of the invention is a method of processing a first material 22 that includes an oil source, and a second material 24 that includes a medium. For the purposes hereof, it will be understood that an “oil source” may be any liquid, solid, or gas (or combination thereof) that may, upon suitable processing thereof (with or without other materials) be transformed into petroleum or a petroleum-related product. The first material 22 may also include the medium (i.e., similar or substantially the same as the medium of the second material 24) or another medium.

Those skilled in the art would appreciate that a wide variety of materials may be suitable as the first material, and also that many materials may be suitable as the second material. For instance, in one embodiment, the first material 22 preferably is raw oil sands material. In another embodiment, the second material 24 preferably is a tailings mixture, taken from a tailings pond that includes tailings from processing the oil sands material, as will be described. The tailings mixture includes tailings solids, and tailings water, as will also be described. Where the second material 24 is the tailings mixture, the medium is sand. Also, where the first material 22 is the raw oil sands material, such first material also includes the medium, i.e., sand.

Alternatively, and as will be described, the first material 22 may be an oil shale material, in which the oil source is kerogen. The second material 24 utilized with the oil shale material in the method of the invention may be any suitable material, e.g., the tailings mixture.

In another alternative embodiment, the first material 22 may be any suitable high carbon material, for instance, petcoke or certain plastics. The second material 24 utilized with the high carbon material in the method of the invention may be any suitable material, e.g., the tailings mixture. As will be described, in another embodiment, the high carbon material may be a third material, added to the blended feedstock mixture with the first and second materials, to increase the carbon content of the blended feedstock mixture. For the purposes hereof, a high carbon material means any material that includes a high proportion of one or more carbon-including compounds therein.

Preferably, a predetermined first amount of the first material 22 and a predetermined second amount of the second material 24 are mixed together in a mixer 32 to provide a blended feedstock mixture 34 that includes predetermined respective proportions of the first material 22 and the second material 24. As will be described, the predetermined respective proportions are determined based, at least in part, on the composition of the first material 22 and the second material 24.

The blended feedstock mixture 34 preferably also includes water. The water may be included in the second material 24, or in the first material 22, or in both.

In a pre-distillation process, the blended feedstock mixture 34 is heated in a pre-distiller 35 to between approximately 100° C. and approximately 150° C., to evaporate water (i.e., to produce steam), and to vaporize light hydrocarbons. As will be described, steam “S0” is produced. For clarity of illustration, the pre-distillation process is schematically represented in the drawings as taking place in a pre-distiller 35. However, as will be described, the pre-distillation process may not necessarily take place in a separate unit of the system.

Subsequently, the blended feedstock mixture 34 is subjected to a distillation process, in which the blended feedstock mixture 34 is heated to between approximately 535° C. and approximately 600° C. to at least partially crack and vaporize the oil source, to provide (i) atmospheric gas oil 36 and (ii) vacuum gas oil 38 from the oil source, (iii) coked medium material 40 that includes carbon-heavy hydrocarbons and the medium (e.g., sand), and (iv) a first barren hot medium material 42. For clarity of illustration, the distillation process is schematically represented in the drawings as taking place in a distiller 31. However, as will be described, the distillation process may not necessarily take place in a separate unit of the system.

It is preferred that the coked medium material 40 is further heated to between approximately 700° C. and approximately 800° C. For clarity of illustration, the post-distillation heating is schematically represented in FIG. 1 as taking place in a post-distiller heater 37. Those skilled in the art would appreciate that the post-distiller heater 37, configured and located to so heat the coked medium material 40 downstream from the distillation process, may be included in the distiller 37, in a gasifier 43, or as a separate unit, as illustrated in FIG. 1.

It will also be understood that the pre-distiller 35, the distiller 31, and the post-distiller heater 37 are schematically illustrated in FIG. 1 as separate units for clarity of illustration. In practice, the pre-distillation process, and the distillation process, and the post-distillation heating of the coked medium material 40 may take place in one or more units, arranged in any suitable manner.

Preferably, the coked medium material 40 is further heated in the gasifier 43 to between approximately 850° C. and approximately 1,000° C., to produce (i) a second dry barren hot medium material 44, and (ii) a syngas 46 that includes hydrogen and carbon monoxide gases. It is also preferred that heat energy is transferred from the first barren hot medium material 42 to the blended feedstock mixture 34 in the pre-distiller 35, as will also be described. In addition, it is also preferred that heat energy is transferred from the second barren hot medium material 44 to the blended feedstock mixture 34 that is in the distiller 31, or the post-distiller heater 37 (FIG. 1). Preferably, one or more of air and oxygen are injected into the gasifier 43, to promote at least partial oxidation of the coked medium material therein.

It will be understood that the coked medium material 40, the first barren hot medium material 42, and the second barren hot medium material 44 may not necessarily be derived solely from the medium of the second material 24. For instance, where the first material 22 is a raw oil sands material, the first material 22 also includes sand, as well as the oil source (i.e., bitumen).

Preferably, at least a portion of the steam “S0” from the pre-distiller 35 is fed to the gasifier 43 in a predetermined amount “S1”, with any excess steam “S2” fed to a separator 48 (FIG. 1). It will be understood that, alternatively, the portion of the steam “S2” may, in whole or in part, be directed to another process.

Those skilled in the art would be aware that water is injected into the gasifier 43. As the operation of gasifiers is well known in the art, further description thereof is unnecessary.

After the first barren hot medium material has been used to transfer heat to the blended feedstock mixture in the pre-distillation process, the first barren hot medium material exits the pre-distillation process substantially inert, i.e., it is suitable for use in reclamation at the mine site.

It is also preferred that heat energy is transferred from one or more of the atmospheric gas oil 36, the vacuum gas oil 38, and the syngas 46 to one or more of the blended feedstock mixture 34, the first barren hot medium material, and the second barren hot medium material. It will be understood that the heat transfer may be effected at any suitable point or points in the system. For example, heat may be transferred to the blended feedstock mixture 34 from one or more of the atmospheric gas oil 36, the vacuum gas oil 38, and the syngas 46 at one or more points in the pre-distillation process and/or the distillation process.

It will also be understood that the atmospheric gas oil 36 and the vacuum gas oil 38 may be extracted at one or more points in the distillation process.

It is believed that the embodiment of the method of the invention described above may be used in processing oil shale, with some modifications. Those skilled in the art would appreciate that “shale oil”, a substitute for crude oil, may be produced from the kerogen in the oil shale.

It will be understood that the method of the invention is directed to processing the raw oil sand material, and/or the oil shale, that has been mined. It will also be understood that because various intermediate steps would be familiar to one skilled in the art (e.g., crushing and screening material at different points in the process), further description thereof is unnecessary.

It will also be understood that, in the drawings, only flows of material are schematically represented, for clarity of illustration. Those skilled in the art would appreciate that heat energy may be transferred from and to a variety of materials and/or mixtures at different points in the method of the invention. For example, heat energy may be transferred from the syngas 46 to the blended feedstock mixture 34 in the pre-distiller 35 or in the distiller 31. Also, heat energy may be transferred from the syngas 46 to the coked oil sands material 40 in the gasifier 43, or to the first and/or second barren hot oil sands material 42, 44.

Those skilled in the art would appreciate that the products of the method 20 preferably are also further processed. For example, in one embodiment, the atmospheric gas oil 36 and the vacuum gas oil 38 preferably are refined to provide refined products such as liquefied naphtha, petroleum gas and gasoline, jet fuel, diesel fuel, and gas oil.

The syngas 46 preferably is further subjected to one or more gas-to-liquid processes (not shown) to provide one or more of gasoline, diesel fuel, methanol, naphtha, and petrochemical feedstock.

As noted above, the blended feedstock 34 preferably is heated in the pre-distillation process. Steam “S0” is produced, as one of the results of the pre-distillation process (FIG. 1). Preferably, the steam “S0” is directed to the gasifier 43 in a predetermined amount “S1”, with any excess steam “S2” being directed to the separator 48. The predetermined amount “S1” is determined based on then current conditions. Those skilled in the art would appreciate that the portion of the steam “S2” may be processed in the separator 48 to preferably provide recovered heat energy (not shown) to be fed to the blended feedstock mixture 34 in the pre-distiller 35. Light hydrocarbons preferably are also recovered in the separator.

As can be seen in FIG. 1, air (or, alternatively, oxygen) (identified in FIG. 1 by reference character “O2”) is injected in a controlled manner into the gasifier 43, to control partial oxidation of the coked medium material 40 within the gasifier 43.

Those skilled in the art would appreciate that, where the second material 24 is tailings resulting from oil sands processing, a substantial proportion of the second material 24 may be water. The water content of the tailings mixture is typically from about 20% by weight, or higher. The water content of the oil sand material from Alberta is typically about 4% by weight.

The water content of the first material 22 may vary, depending on the source thereof. Those skilled in the art would appreciate that oil sands found at other locations (e.g., Utah) may typically include different water content. For example, the water content of the raw oil sands material found in Utah is approximately zero. Also, the water content of oil shale is approximately zero.

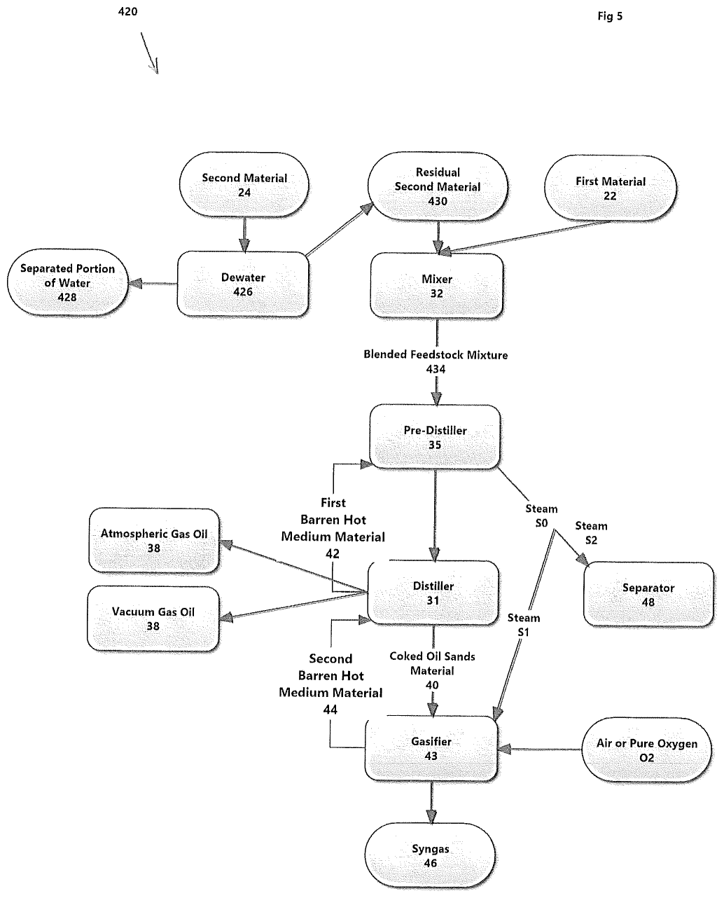

As will be described, because of the relatively large amount of water in the tailings mixture 24, in one embodiment, the process may include an initial step of dewatering the tailings mixture 24 that is to be processed (FIG. 5).

Those skilled in the art would appreciate that the tailings mixture includes tailings solids (not shown) which have a hydrocarbon content (i.e., the residual bitumen) that is significantly lower than the hydrocarbon content of the raw oil sands material. Accordingly, the tailings mixture 24 is low-carbon material, relative to the raw oil sands material.

It will be understood that the composition and grain size of the raw oil sands material (i.e., the first material 22) and of the tailings mixture (i.e., the second material 24) that are utilized in the method of the invention may be highly variable, even over a relatively short period of time. For example, an average composition of the raw oil sands mixture from Alberta, Canada is as follows:

| quartz, silt, silica sand | 50-60% by mass |
|---|---|
| clay | 10-30% |
| water | 3-6% |
| bitumen | 5-15% |

The tailings ponds, after the tailings have been allowed to settle, typically include layers of different materials. The layers result from the settling of the tailings mixture over time. Those skilled in the art would appreciate that, in practice, the layers of the tailings pond can become mixed together when an amount of the tailings mixture is taken from the tailings pond. It can be seen, therefore, that the tailings mixture as actually provided in the initial step of the process 20 herein may have variable characteristics, depending on where in the tailings pond the amount of the tailings mixture that is being processed is taken from.

The layers in the settled tailings pond are typically divided into four separate layers. The uppermost of the four layers in the settled tailings pond is referred to as “free water”, or “reclaimed water”. The free water includes negligible sand, fines, and bitumen. In the prior art, the free water may be recycled for use in the bitumen extraction process.

Fine tailings solids, defined as particles less than 44 micrometers in diameter, mostly tend to settle into three of the four layers of the tailings pond, below the layer of free water. Immediately underneath the layer of free water is a layer of “fine fluid tailings”, described further below. Next, immediately underneath the fine fluid tailings is a third layer, consisting mostly of “mature fine tailings”.

The fourth, and vertically the lowest layer, includes coarse tailings solids.

Those skilled in the art would appreciate that, when the tailings mixture from the oil sands processing plant is first delivered into the tailings pond containment area, the coarse sand sinks to the bottom, and a proportion of the fine tailings solids in that mixture is trapped at that point between the coarse tailings solids particles. The coarse tailings pond material typically contains relatively small amounts of bitumen.

The balance of the fine tailings solids are suspended in the tailings pond water, or form the fine fluid tailings or the mature fine tailings. The mature fine tailings are described as “sludge-like materials”. As noted above, the fine fluid tailings form the layer in the tailings pond that is immediately below the layer of free water. The mature fine tailings form the layer in the tailings pond that is between the fine fluid tailings and the coarse tailings solids.

The difference between the fine fluid tailings and the mature fine tailings is that the mature fine tailings have been aged. If the fine fluid tailings are left undisturbed for several years, then the fine fluid tailings become mature fine tailings.

Those skilled in the art would appreciate that, in practice, the tailings mixture is created when an amount of the tailings is removed from the tailings pond. Layers of the tailings may be mixed together at that time. As noted above, in one embodiment, the predetermined first amount of the raw oil sands material preferably is mixed with the predetermined second amount of the tailings mixture to form the blended feedstock mixture 34.

It will be understood that the minimum proportion for each layer of the tailings mixture in the blended feedstock mixture 34 is zero in each case. Also, the minimum predetermined second amount of the tailings mixture is zero. (As described above, in an initial step of one embodiment, the predetermined first amount of the raw oil sands material preferably is mixed with the predetermined second amount of the tailings mixture.)

For the purposes hereof, the “fines” are considered to be the second and third layers (i.e., the layers of the fine fluid tailings (second layer) and the mature fine tailings (third layer)) of the settled tailings pond, described above. It will also be understood that the coarse tailings layer (i.e., the lowest, fourth, layer) may include fine tailings that are “trapped” in the layer of coarse tailings.

It will be understood that the tailings mixture is a relatively low carbon material, i.e., the carbon content of the tailings mixture is relatively low, compared to that of the raw oil sands. As will be described, in one embodiment, high-carbon material (also hereinafter referred to as a third material) may be added at any suitable point in the method of the invention, to address a carbon shortfall or deficiency in the blended feedstock mixture 34.

As noted above, it is preferred that the method 20 includes a step of recovering heat from water evaporation should there be excess steam “S2”, which depends on the water content of the blended feedstock mixture 34. This step, which involves recovering heat energy from the steam “S0” generated in the pre-distillation process, and transferring the recovered heat energy to the blended feedstock mixture 34, is preferred (where the second material is the tailings mixture) because of the relatively high water content of the tailings mixture. As can be seen in FIG. 1, the method preferably includes directing the steam “S2” through the separator 48, and then transferring the recovered heat energy to the blended feedstock mixture 34 in the pre-distiller 35.

From the foregoing, it can be seen that the amount of the tailings mixture that may be included in the blended feedstock mixture may vary. As a practical matter, in some cases, the predetermined second amount of the tailings mixture that is delivered to the mixer 32 may itself be a mixture of the four layers of the settled tailings mixture described above.

In one embodiment, the separator 48 preferably is a condenser heat exchanger. Those skilled in the art would appreciate that, if there is sufficient steam “S0”, then at least a portion of the steam “S0” directed thereto condenses in the separator 48, to result in distilled water and recovered heat energy. (This option is not illustrated in the drawings, to simplify the drawings.) The recovered heat energy preferably is re-introduced to the blended feedstock mixture 34 in the pre-distiller 35, or may be utilized elsewhere in the process. Depending on the quality of the distilled water, the heat-depleted water may be returned to the tailings pond, injected underground, fed into the gasifier 43, or be otherwise utilized. As noted above, another portion of the steam “S2” preferably is transferred from the pre-distiller 35 to the gasifier 43, or utilized elsewhere.

The coked medium material 40 may be crushed and screened as necessary prior to its introduction into the gasifier 43.

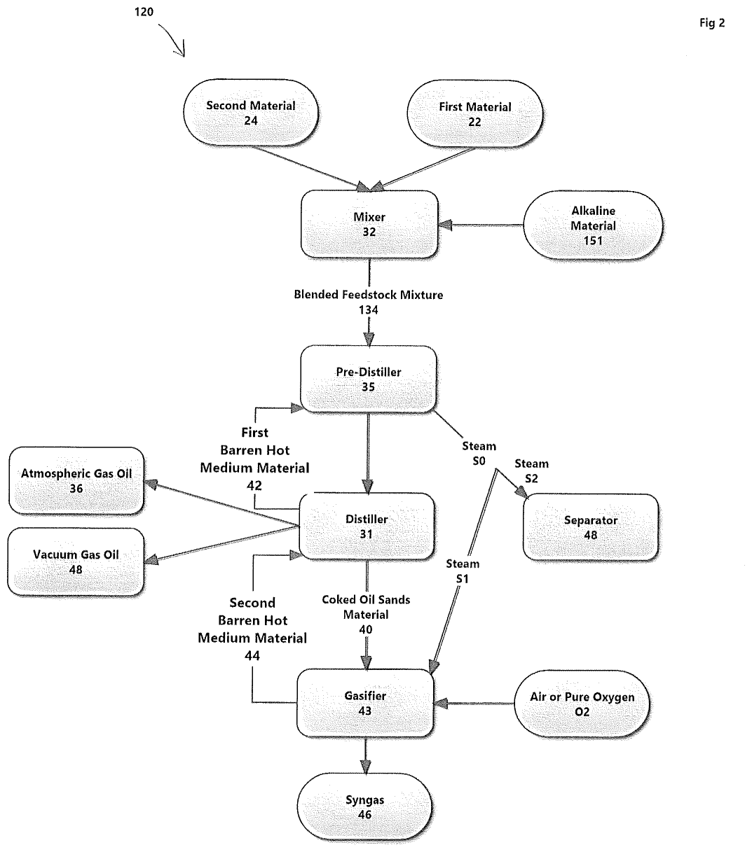

It will be understood that, in certain circumstances, acids may be formed in non-trivial quantities in the processes of the invention, which may cause operational and product output problems. Also, it may be desirable to capture and remove sulphur from the processes of the invention. Accordingly, in an alternative embodiment of the method 120 of the invention, schematically illustrated in FIG. 2, a predetermined amount of alkaline material 151 preferably is added into the mixer 32, and mixed into the blended feedstock mixture 34 to provide a modified blended feedstock mixture 134.

Alternatively, the alkaline material 151 may be added into the gasifier 43. It will be understood that the addition of the alkaline material into the gasification process is omitted from the drawings for clarity of illustration.

Those skilled in the art would appreciate that the predetermined amount of the alkaline material 151 that is added preferably is sufficient to minimize the amounts of acid formed in the processes 120 of the invention. The alkaline material 151 may be any suitable material. For example, the alkaline material may be limestone, which reacts with the sulphur to produce gypsum.

It will be understood that, except for the addition of the alkaline material 151 and the modified blended feedstock material 134, the method 120 is substantially the same as the method 20 of the invention.

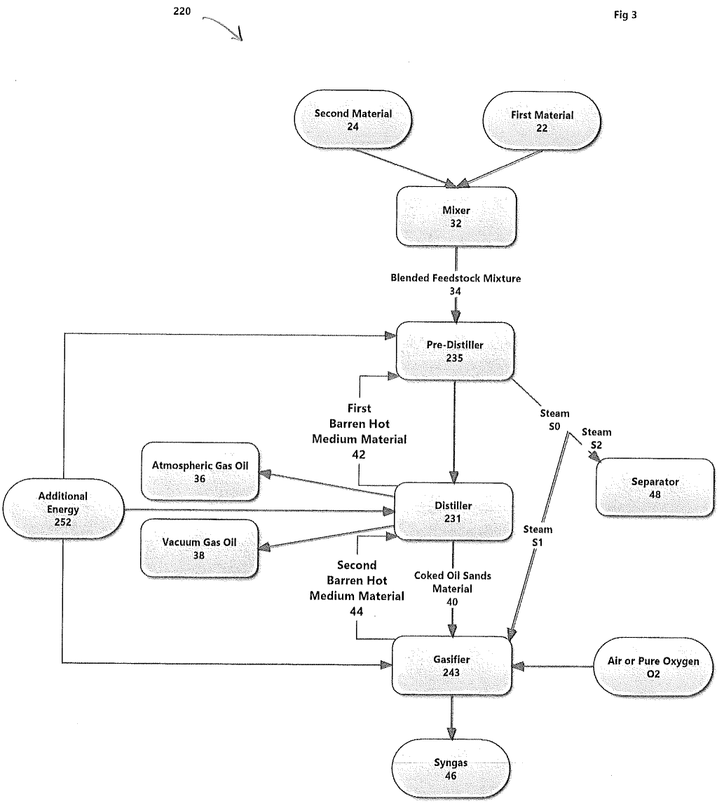

In certain conditions (e.g., at start-up, at shutdown, or when the blended feedstock mixture includes excessive moisture), additional heat energy may be required to be added, at certain points in the method of the invention, as needed. It will be understood that additional energy inputs may be supplied via any suitable additional energy source 252. For example, the additional energy input may be provided via combustion of a fuel (e.g., natural gas, oil, coal, petcoke, hydrogen) or from any other suitable energy source. Accordingly, in one embodiment of the method 220 of the invention (FIG. 3), natural gas 252 preferably is burned to provide heat in the pre-distillation process in the pre-distiller 235, to further heat the blended feedstock mixture therein.

In another embodiment, the additional energy source 252 preferably is utilized to provide heat in the distillation process in the distiller 231, to further heat the blended feedstock mixture therein. It will be understood that heat energy may be added into the post-distiller heater, and/or the gasifier as well. Those skilled in the art would appreciate that burning the natural gas 252 to add heat energy into the distillation process is only one way to add heat energy to the processes 220 of the invention. In this embodiment, as shown in FIG. 3, the partial or complete oxidation of the natural gas preferably also takes place in the gasifier 243 as needed to add heat and to improve syngas quality. Other ways to add additional energy may be, for example, burning one or more of oil, coal, syngas, petcoke, hydrogen, or utilizing any other suitable source of heat energy.

Those skilled in the art would also appreciate that the method 220 of the invention may include any suitable combination of the foregoing arrangements. For instance, the natural gas (or any other suitable source of additional energy) 252 may be utilized in all or some of the pre-distiller 235, the distiller 231, the post-distiller heater, and the gasifier 243, or alternatively, the natural gas may be utilized in any one or more of them. Preferably, the system is arranged to permit and to control utilization of the natural gas 252 in all or any one or more of the pre-distiller 235, the distiller 231, the post-distiller heater, and the gasifier 243, in order to achieve optimum performance, in view of conditions that may vary, and consequently may require adjustments to be made. Depending on the conditions, no additional energy source 252 may be required at times.

It will be understood that, except for the addition of the additional heat energy at one or more of various stages in the processes of the invention, the method 220 is substantially the same as the method 20 schematically illustrated in FIG. 1. The post-distiller heater has been omitted from FIG. 2 for clarity of illustration. As noted above, the post-distiller heater may be included in the distiller 31 or the gasifier 43.

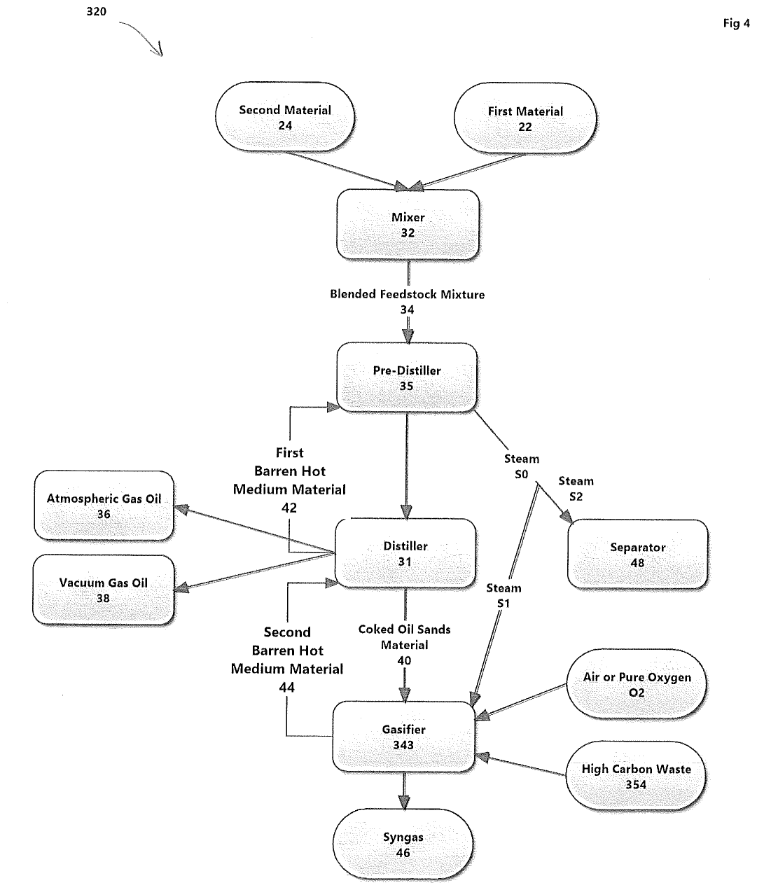

As noted above, the tailings mixture has a relatively low carbon content. Accordingly, in alternative embodiments, relatively high carbon material (the third material) may be added into the processes of the invention, where suitable, to increase heat energy therein. This may be achieved in different ways. For example, in the embodiment of the method 320 of the invention schematically illustrated in FIG. 4, the high-carbon material 354 may be oxidized in the gasifier (FIG. 4), to improve the syngas.

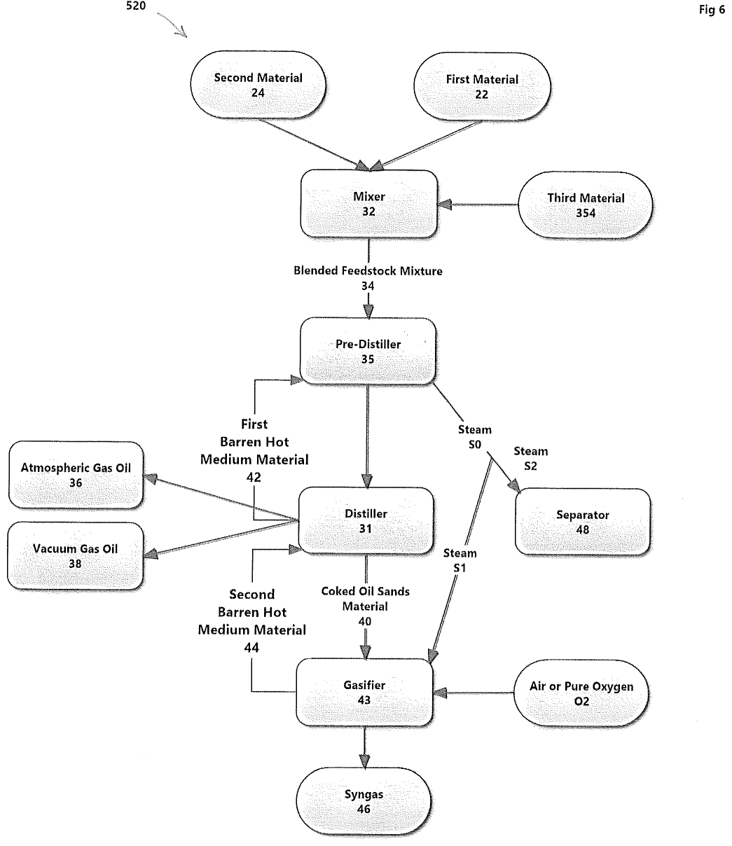

As another example, and as can be seen in FIG. 6, in one embodiment of the method 520 of the invention, high-carbon material 354 (identified in FIG. 6 as a third material) preferably is added to the blended feedstock mixture, to increase the carbon content thereof.

It will be understood that, except for the addition of the high-carbon material 354 to the blended feedstock mixture, the method 520 is substantially the same as the method 20 schematically illustrated in FIG. 1. The post-distiller heater has been omitted from FIG. 6 for clarity of illustration. As noted above, the post-distiller heater may be included in the distiller 31 or the gasifier 43.

Such high-carbon material may be any suitable material. For instance, the high-carbon material may be petcoke, or any suitable plastic material. It is believed that the high-carbon material can provide required process heat energy for low bitumen oil sands, and/or enable an increase in the tailings percentage in the blended feedstock mixture, and/or improve syngas quality.

As noted above, the bitumen content of the raw oil sands may be found to be less than expected, or less than optimal for known processing technologies. In the method of the invention, however, a lower bitumen content may be addressed by adding a suitable high-carbon material into the process, as described above. Accordingly, in another embodiment of the method 320 of the invention (FIG. 4), high-carbon material 354 (i.e., material with relatively high carbon content, relative to the raw oil sands material 22), such as petroleum coke (“petcoke”) or coal, is preferably added to the coked oil sands material 40 in a gasifier 343.

It will be understood that, except for at least partial oxidation of the petcoke in the gasifier 343, the method 320 is substantially the same as the method 20 schematically illustrated in FIG. 1. The post-distiller heater is omitted from FIG. 4 for clarity of illustration. As noted above, the post-distiller heater may be included in the distiller 31 or the gasifier 43.

It will also be understood that, in yet another alternative embodiment, the raw oil sands material and the high-carbon material 354 may be mixed together, in the absence of the tailings mixture, to provide a high-carbon feedstock mixture. In this embodiment, the first material is the raw oil sands material, and the second material is the high-carbon material. This may be advantageous, for example, in circumstances in which an excess of the high-carbon material is required to be processed, or for processing raw oil sands material with relatively low carbon content.

It will also be understood that the method of the invention may function with a blended feedstock mixture that includes high-carbon material and the tailings mixture, i.e., the amount of the raw oil sands in the blended feedstock mixture may be zero. In this embodiment, the first material is the high-carbon material, and the second material is the tailings mixture.

An alternative method 420 of the invention is schematically illustrated in FIG. 5. As noted above, depending on the amount of water in the tailings mixture prior to forming the blended feedstock mixture, the tailings mixture may be subjected to dewatering, before the blended feedstock mixture is formed (FIG. 5). In one embodiment, the tailings mixture 24 preferably is subjected to a dewatering process 426 to separate a separated portion 428 of the tailings water and a residual tailings mixture 430. Those skilled in the art would appreciate that, although some of the tailings water is removed by dewatering to form the separated portion 428 of the tailings water, the residual tailings mixture 430 also includes an unseparated portion of the tailings water.

A predetermined second amount of the residual tailings mixture 430 preferably is mixed in the mixer 32 with a predetermined first amount of the raw oil sands material 22 to provide the blended feedstock mixture 434. The blended feedstock mixture 434 preferably is processed in substantially the same manner as the blended feedstock mixture is then processed in the other embodiments of the method of the invention, described above.

It will be understood that, except for the dewatering of the second material 24, the method 420 is substantially the same as the method 20 schematically illustrated in FIG. 1. The post-distiller heater is omitted from FIG. 5 for clarity of illustration. As noted above, the post-distiller heater may be included in the distiller 31 or the gasifier 43.

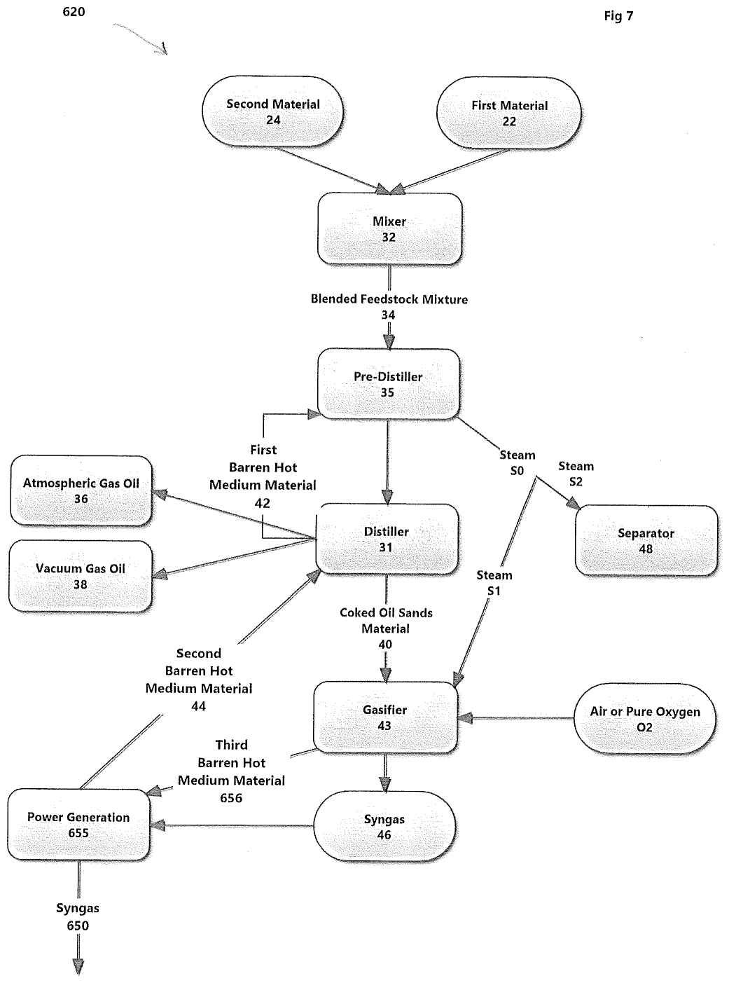

In yet another embodiment of the method 620 of the invention, schematically illustrated in FIG. 7, in a power generation unit 655, the thermal energy from the hot barren oil sands and/or the syngas is utilized to generate power (e.g., electricity). Alternatively, or in addition, syngas may also be combusted in the power generation unit 655, to generate power. It will be understood that, except for the utilization of the syngas and a third barren hot medium material 656 in power generation, the method 620 is substantially the same as the method 20 schematically illustrated in FIG. 1. Preferably, the generated power is used to power process work and electrical requirements such as pumping, motors, lighting, sensors, mixing, material transport, and crushing.

As can be seen in FIG. 7, the third barren hot medium material 656 preferably is produced by the gasifier 43 and heat energy therefrom is used to generate power. Subsequently, after heat transfer from the third barren hot medium material, the barren hot medium material (identified in FIG. 7 for clarity as the second barren hot medium material 44), which retains a certain amount of heat energy at that point, is directed to the distiller 31, for transfer of heat energy therefrom to the part of the blended feedstock mixture 34 that is then in the distiller 31 (FIG. 7).

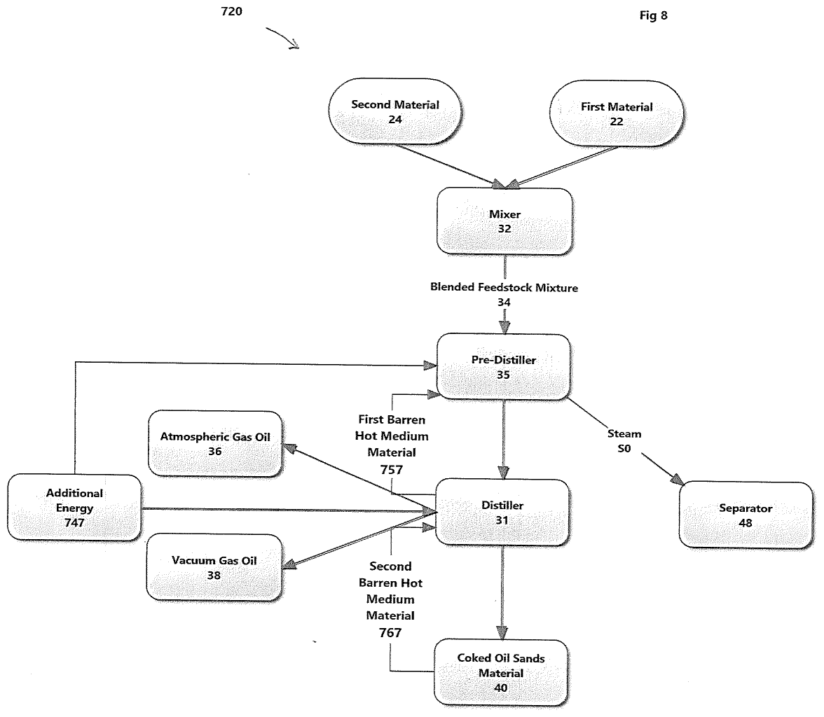

In yet another embodiment of the method 720 of the invention, the gasifier is omitted, either physically or as an operation mode. In this embodiment hot coked medium material 40, at between approximately 350° C. and approximately 600° C., is directly returned to the distiller 31. In FIG. 8, the hot coked medium material that is returned to the distiller 31 is identified as a second hot coked medium material 767, for clarity. In this embodiment, the distiller 31 produces a first hot coked medium material 757, which preferably is directed to the pre-distiller 35, for heat transfer to the blended feedstock mixture 34 in the pre-distiller 35. Further, with no gasifier, all the steam “S0” is directed to the separator 48. Without the gasifier the additional heat energy required for pre-distillation and distillation would preferably come from the burning of natural gas in heater 747, but other additional energy sources may be utilized as well, or instead of natural gas.

It will be understood that the initial step in the method 720 of mixing the first amount of the raw oil sands material 22 and the second amount of the tailings mixture 24 to produce the blended feedstock mixture 34 is substantially the same as the corresponding step in the method 20 schematically illustrated in FIG. 1.

It will also be understood that, except for the omission of the gasifier, the method 720 is substantially the same as the method 20 schematically illustrated in FIG. 1. The post-distiller heater is omitted from FIG. 8 for clarity of illustration. As noted above, the post-distiller heater may be included in the distiller 31.

Those skilled in the art would appreciate that the method of processing the raw oil sands 22 in the absence of the gasifier may occur in practice from time to time in a variety of circumstances, e.g., if the gasifier is down, (e.g., for maintenance), or if there is a desire to eliminate tailings and acceptance of only producing light hydrocarbons, atmospheric gas oil, and vacuum gas oil. Preferably, the cooled coked oil sands material are re-introduced subsequently to the process with a gasifier at either the mixer or the gasifier, in a preselected amount.

Those skilled in the art would also appreciate that the steps of different embodiments of the method of the invention may be combined from time to time, as may be necessary or desirable. For example, the dewatering process may be utilized where the alkaline material is also added to form the blended feedstock material, and natural gas and/or petcoke may also be utilized in a method including the dewatering process and/or the addition of the alkaline material. Similarly, in an embodiment of the method that includes a dewatering step and/or adding the alkaline material, the additional energy source may also be utilized, as appropriate.

In addition, it will be understood that the embodiments of the method of the invention described above may be utilized to process “high-carbon” material (including, but not limited to, petcoke and/or suitable plastics), as described above. Similarly, the method of the invention may be utilized to process the raw oil sands material and the oil shale material with such modifications as may be necessary or desirable.

It will be appreciated by those skilled in the art that the invention can take many forms, and that such forms are within the scope of the invention as claimed. The scope of the claims should not be limited by the preferred embodiments set forth in the examples, but should be given the broadest interpretation consistent with the description as a whole.

We claim:

1. A method of processing a first material comprising an oil source and a second material comprising a medium, the method comprising:
(a) mixing the first material and the second material to provide a blended feedstock mixture comprising predetermined respective proportions of the first material and the second material, the blended feedstock mixture comprising water;
(b) in a pre-distillation process, heating the blended feedstock mixture to between approximately 100° C. and approximately 150° C., to produce steam from the water and to vaporize light hydrocarbons from the oil source;
(c) in a distillation process, further heating the blended feedstock mixture to between approximately 535° C. and approximately 600° C. to at least partially crack and vaporize the oil source, to provide (i) atmospheric gas oil and (ii) vacuum gas oil from the oil source, (iii) a coked medium material comprising carbon-heavy hydrocarbons and the medium, and (iv) a first barren hot medium material;
(d) heating the coked medium material to between approximately 700° C. and approximately 800° C.;
(e) in a gasification process, heating the coked medium material to between approximately 850° C. and approximately 1,000° C., to produce a second dry barren hot medium material and syngas comprising hydrogen and carbon monoxide gases;
(f) transferring heat energy from at least a portion of the first barren hot medium material to the blended feedstock mixture in the pre-distillation process;
(g) transferring heat energy from at least a portion of the second barren hot medium material to the blended feedstock mixture in the distillation process; and
(h) injecting at least one of air and oxygen into the gasification process, to promote at least partial oxidation of the coked medium material therein.

2. A method according to claim 1 in which the first material is a raw oil sands material and the oil source is bitumen.

3. A method according to claim 2 in which the second material is a tailings mixture.

4. A method according to claim 1 in which the first material is an oil shale material and the oil source is kerogen.

5. A method according to claim 4 in which the second material is a tailings mixture.

6. A method according to claim 1 in which the first material is a high carbon material comprising at least one carbon-including compound, and the oil source is said at least one carbon-including compound.

7. A method according to claim 6 in which the second material is a tailings mixture.

8. The method according to claim 1 additionally comprising transferring heat energy from at least one of the atmospheric gas oil, the vacuum gas oil, and the syngas to at least one of the blended feedstock mixture, the first barren hot medium material, and the second barren hot medium material.

9. The method according to claim 1 additionally comprising refining the atmospheric gas oil and the vacuum gas oil to provide at least one of liquefied naphtha, petroleum gas, and gasoline, diesel fuel, jet fuel, and gas oil.

10. The method according to claim 1 in which the syngas is further subjected to at least one gas-to-liquid process to provide at least one of gasoline, diesel fuel, methanol, naphtha, and petrochemical feedstock.

11. The method according to claim 1 additionally comprising, in the mixer, mixing a preselected amount of an alkaline material into the blended feedstock mixture, to provide a modified blended feedstock mixture.

12. The method according to claim 1 additionally comprising utilizing an additional energy source in the pre-distillation process, to further heat the blended feedstock mixture therein.

13. The method according to claim 1 additionally comprising utilizing an additional energy source in the distillation process, to further heat the blended feedstock mixture.

14. The method according to claim 1 additionally comprising at least partially oxidizing high carbon material to provide heat energy in the gasification process.

15. The method according to claim 1 additionally comprising, in the mixer, mixing a predetermined amount of high carbon material into the blended feedstock mixture, to increase the carbon content of the blended feedstock mixture.

16. The method according to claim 1 in which the heat energy from at least one of first barren hot medium material, the second barren hot medium material, and the syngas is utilized to generate electricity.

17. The method according to claim 16 in which the syngas is combusted in order to generate electricity.

18. A method of processing a first material comprising an oil source and a second material comprising a medium and water, the method comprising:
(a) subjecting the second material to a dewatering process to separate a separated portion of the water therefrom, to provide the separated portion of the water and a residual second material comprising at least a portion of the second material and an unseparated portion of the water;
(b) mixing a predetermined first amount of the first material and a predetermined second amount of the residual second material to provide a blended feedstock mixture;
(c) in a pre-distillation process, heating the blended feedstock mixture to between approximately 100° C. and approximately 150° C., to produce steam;
(d) in a distillation process, heating the blended feedstock mixture to between approximately 535° C. and approximately 600° C. to at least partially crack and vaporize the oil source, to provide (i) atmospheric gas oil and (ii) vacuum gas oil from the oil source, (iii) a coked medium material comprising carbon-heavy hydrocarbons and the medium, and (iv) a first barren hot medium material;
(e) transferring heat energy from the steam to the blended feedstock mixture in the pre-distillation process;
(f) heating the coked medium material to between approximately 700° C. and approximately 800° C.;
(g) in a gasification process, heating the coked medium material to between approximately 850° C. and approximately 1,000° C., to produce a second dry barren hot medium material and syngas comprising hydrogen and carbon monoxide gases;
(h) transferring heat energy from at least a portion of the first barren hot medium material to the blended feedstock material in the pre-distillation process;
(i) transferring heat energy from at least a portion of the second barren hot medium material to the blended feedstock mixture in the distillation process; and
(j) injecting at least one of air and oxygen into the gasification process.
